(19) United States
(12) Patent Application Publication
(10) Pub. No.: US 2025/0006319 A1
(43) Pub. Date: Jan. 2, 2025

(54) SYSTEMS AND METHODS FOR VETERINARY ORAL CARE HEALTH TEST

(71) Applicant: Mars, Incorporated, McLean, VA (US)

(72) Inventors: Marie-Louise Amanda BENNETT, Melton Mowbray (GB); Gordon Craig CAMERON, Franklin, TN (US); Lucy Jane HOLCOMBE, Melton Mowbray (GB); Philip Martin MCGENITY, Melton Mowbray (GB); Avika Kishorlal RUPARELL, Melton Mowbray (GB); Corryn Victoria WALLIS, Melton Mowbray (GB); Amanda Elizabeth DAVIES, Melton Mowbray (GB)

(21) Appl. No.: 18/709,166

(22) PCT Filed: Nov. 11, 2022
(86) PCT No.: PCT/US2022/079684

§ 371 Date: May 10, 2024

Related U.S. Application Data
(60) Provisional application No. 63/278,693, filed on Nov. 12, 2021.

(51) Int. Cl. G16H 10/40; G16H 50/20; G16H 50/30

(52) U.S. Cl. CPC G16H 10/40; G16H 50/20; G16H 50/30

(57) ABSTRACT

Methods and systems are disclosed for providing pet health diagnostic and aftercare information. Pet metadata associated with an activation code is received from a pet profile server, wherein the pet metadata and the activation code have been input by a first user using a first user device. Quantitative polymerase chain reaction (qPCR) results associated with the activation code are then received from a qPCR results server. A correlation is determined between the pet metadata and the qPCR results based on the activation code. One or more reports containing a diagnosis and/or a health recommendation are generated based on processing the pet metadata and the qPCR results. The generated one or more reports are transmitted to at least the pet profile server, the first user device, or a veterinarian device for display.

### CROSS-REFERENCE TO RELATED APPLICATION

This is a National Stage Application under 35 U.S.C. § 371 of International Application No. PCT/US2022/079684, filed on Nov. 11, 2022, which claims priority to U.S. Patent Application No. 63/278,693, filed on Nov. 12, 2021, the entireties of which are incorporated herein by reference.

### TECHNICAL FIELD

Various embodiments of the present disclosure relate generally to pet health assessment and, more particularly, to systems and methods for providing pet health diagnosis and aftercare information based on associating pet metadata and results of an oral diagnostic kit obtained from disparate sources.

### BACKGROUND

One of the leading causes of disease in household pets is poor oral hygiene. When left untreated, symptoms of bad breath, tartar buildup, and cavities in pets can often lead to gum disease (e.g., periodontal disease), and even more serious health issues, such as oral cancer, as well as heart, kidney, and liver conditions. However, with early detection and proper treatment, gum disease and related diseases in pets are preventable.

While veterinarians diagnose and treat gum disease as well as symptoms thereof, the disease remains underdiagnosed in pets. Methods of predicting and/or diagnosing gum disease in pets include visual oral examination, costly anaesthetized procedures, and the evaluation of risk factors. However, visual examinations and predictive modeling based on risk factors are limited in scope and often require supplemental analysis. Other methods have been undertaken to study a pet's oral microbiome, such as the analysis of a collected DNA sample.

Even when veterinarians diagnose and treat oral health conditions, pets remain at risk for these conditions due to pet owners' failure to maintain proper aftercare and infrequent veterinarian visits. Therefore, a need exists for a personalized system and method for providing information regarding the diagnosis, treatment, and/or prevention of oral health conditions in a pet, in a manner that allows collaboration between a pet owner and a veterinarian.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for providing pet health diagnostic and aftercare information based on analyzed data in association with an activation code of an oral diagnostic kit. The analyzed data may include i) pet metadata associated with a pet, e.g., pet metadata input by a user device after activating the activation code of the oral diagnostic kit and ii) test results from testing an oral sample from the pet collected by a user via the oral diagnostic kit.

In one aspect, an exemplary system for providing pet health diagnostic and aftercare information may include a first user device, a second user device, a pet profile server, a test results server, and an analytics component. The pet profile server may include a pet profile storage. The pet profile server may be configured to receive an activation code from the first user device, receive pet metadata from at least one of the first user device or an electronic medical record (EMR) data storage, and store the pet metadata in association with the activation code in the pet profile storage. The test results server may include a test results storage. The test results server may be configured to receive the activation code from the second user device, receive test results data from the second user device, and store the test results data in association with the activation code in the test results storage. The analytics component may be executed by one or more processors. The analytics component may be configured to receive the pet metadata associated with the activation code from the pet profile server and receive the test results data associated with the activation code from the test results server and upon determining a correlation between the pet metadata and the test results data based on the activation code, process the pet metadata with the test results data. The analytics component may be configured to generate at least one diagnosis and/or at least one health recommendation based on the processed data and to generate one or more reports based on the at least one diagnosis and/or the at least one health recommendation.

In another aspect, an exemplary system for providing pet health diagnostic and aftercare information may include a pet profile database storing pet metadata in association with an activation code from an oral diagnostic kit, a quantitative polymerase chain reaction (qPCR) results database storing qPCR results in association with the activation code, and an analytics component. The analytics component may be executed by one or more processors. The analytics component may be configured to retrieve the pet metadata associated with the activation code from the pet profile database, retrieve the qPCR results associated with the activation code from the qPCR results database, and upon determining a correlation between the pet metadata and the qPCR results based on the activation code, process the pet metadata with the qPCR test results. The analytics component may be configured to generate at least one diagnosis and/or at least one health recommendation based on the processed data and to generate one or more reports based on the at least one diagnosis and/or the at least one health recommendation.

In yet another aspect, an exemplary computer implemented method of providing pet health diagnostic and aftercare information may include receiving, from a pet profile server, which may include a pet profile storage, pet metadata associated with an activation code, wherein the pet metadata and the activation code may be input by a first user using a first user device. The activation code may be identified by the first user from an oral diagnostic kit. The method may include receiving, from a quantitative polymerase chain reaction (qPCR) results server, which may include a qPCR results storage, qPCR results associated with the activation, wherein the qPCR results are obtained from a qPCR assay of an oral sample collected from a pet. Upon determining a correlation between the pet metadata and the qPCR results based on the activation code, the method may include processing the pet metadata with the qPCR results. The method may include generating, based on the processed data, one or more reports containing at least one diagnosis and/or at least one health recommendation and transmitting the one or more generated reports to at least one of the pet profile server, the first user device, or a veterinarian device.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts a block diagram of an exemplary system for processing data associated with a pet, according to one or more embodiments.

FIG. 2 depicts an exemplary oral diagnostic kit, according to one or more embodiments.

FIG. 3 depicts a flowchart illustrating an exemplary process for generating user reports based on pet metadata and qPCR results obtained from disparate sources, according to one or more embodiments.

FIG. 4 depicts a flowchart illustrating an exemplary method of receiving and displaying a report on a user device, according to one or more embodiments.

FIG. 5 depicts a flowchart illustrating exemplary steps performed by a pet profile server, according to one or more embodiments.

FIG. 6 depicts a flowchart illustrating exemplary steps performed by a qPCR results server, according to one or more embodiments.

FIG. 7 depicts a block diagram illustrating a linkage between pet metadata and qPCR results received from a pet profile server and a qPCR results server respectively, according to one or more embodiments.

FIG. 8 depicts a flow chart illustrating exemplary steps performed by an analytics component, according to one or more embodiments.

FIG. 9 illustrates an implementation of a computer system that may execute techniques presented herein.

### DETAILED DESCRIPTION

Various embodiments of the present disclosure relate generally to pet health assessment and, more particularly, to systems and methods for providing pet health diagnosis and aftercare information based on associating pet metadata and results of an oral diagnostic kit obtained from disparate sources.

As discussed above, poor oral hygiene in pets may lead to serious overall health and oral health conditions, such as periodontal disease. However, diseases such as periodontal disease are largely underdiagnosed in pets, due to the uncertainties associated with visual examination and predictive modeling. Other more reliable diagnosis and treatment methods, such as anaesthetized procedures may not be pursued due to costliness. While various methods of assessing a pet's oral microbiome exist, these methods fail to utilize a comprehensive list of factors contributing to a pet's oral health. Further, even when a veterinarian provides a pet oral health diagnosis and/or treatment, the pet's health may worsen or regress as a result of improper and/or infrequent aftercare from the pet owner.

Therefore, the embodiments of the present disclosure are directed to solving, mitigating, or rectifying the above-mentioned issues by providing information regarding the diagnosis, treatment, and/or prevention of oral health conditions in a pet, in a cost-effective manner that allows collaboration between a pet owner and a veterinarian, and wherein the information is available to both the pet owner and the veterinarian. The systems and methods of the present disclosure may address the above-mentioned issues by allowing a user such as a pet owner and/or a veterinarian to create a pet profile for a pet based on an activation code from an oral diagnostic kit. Once the oral diagnostic kit has been activated and the pet profile has been created, the user can input a variety of pet metadata related to the pet (e.g., weight, age, and oral care routine) on the pet profile and collect an oral sample from the pet using the oral diagnostic kit. Embodiments of the present disclosure allow the veterinarian to supplement pet metadata input by the pet owner and vice versa. Once the collected oral sample is tested at a laboratory, the test results are linked with the pet metadata for the pet via the activation code from the oral diagnostic kit and analyzed to generate reports with diagnostic and/or aftercare information for the pet owner and the veterinarian. The present disclosure allows for an accurate and cost-effective assessment of pet health and the provision of a personalized oral care plan for both veterinarians and pet owners using a streamlined approach.

The present disclosure provides systems, methods, and/or devices configured to receive and process i) pet metadata associated with an activation code of an oral diagnostic kit and ii) test results from a pet oral sample associated with the activation code of the oral diagnostic kit, and to generate and transmit reports based on the processed data. The following embodiments describe systems and computer implemented methods for providing pet health diagnostic and aftercare information, and more particularly pet oral health diagnostic and aftercare information. The pet health diagnostic and aftercare information is based on data processed by an analytical component containing one or more processors. The data includes pet metadata input via user device on a pet profile server by at least one user after obtaining an oral diagnostic kit and activating the kit as well as test results data from lab testing an oral sample collected from the pet by the user with the oral diagnostic kit.

Specifically, the at least one user who inputs the pet metadata may be a pet owner and/or a veterinarian. A lab technician may conduct the test on the collected oral sample and may input, using a device, the test results and the activation code associated with the collected sample onto a test results server. The pet health diagnostic and aftercare information provided by the systems and methods of the present disclosure may be in the form of one or more reports and may be transmitted to a pet owner and/or a veterinarian. The report for a veterinarian may aid in an oral health diagnosis by providing a risk level for periodontal disease. The report for the pet owner may provide aftercare recommendations for treatment and/or prevention based on the pet's analyzed risk level for periodontal disease.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In the detailed description herein, references to “embodiment,” “an embodiment,” “one non-limiting embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

In general, terminology can be understood at least in part from usage in context. For example, terms, such as “and”, “or”, or “and/or,” as used herein can include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, can be used to describe any feature, structure, or characteristic in a singular sense or can be used to describe combinations of features, structures or characteristics in a plural sense. Similarly, terms, such as “a,” “an,” or “the,” again, can be understood to convey a singular usage or to convey a plural usage, depending at least in part upon context. In addition, the term “based on” can be understood as not necessarily intended to convey an exclusive set of factors and can, instead, allow for existence of additional factors not necessarily expressly described, again, depending at least in part on context.

As used herein, the terms “comprises,” “comprising,” “includes,” “including,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

The terms “pet” and “household pet” as used in accordance with the present disclosure can refer to, without limitation, domesticated or tamed animals such as, e.g., dogs, cats, rabbits, horses, and the like.

The term “pet owner” may include, for example, without limitation, any person, organization, and/or collection of persons that owns and/or provides food and shelter for a pet. For example, a “pet owner” may include a pet adopter, a pet caretaker, a pet caregiver, and an animal shelter.

The term “veterinarian” may include, for example, without limitation, any person, organization, and/or collection of persons that provides medical care to a pet. For example, a “veterinarian” may include a veterinary technician, a veterinary personnel, and a veterinarian practitioner.

The term “lab employee” may include, for example, without limitation, any person and/or collection of persons employed by a laboratory. For example, a “lab employee” may include a lab technician, a scientist, and a researcher.

The terms “canine” and “dog” may include, for example, without limitation, recognized dog breeds (some of which may be further subdivided). For example, the recognized dog breeds may include afghan hound, Airedale, Akita, Alaskan malamute, basset hound, beagle, Belgian shepherd, bloodhound, border collie, border terrier, borzoi, boxer, bulldog, bull terrier, cairn terrier, Chihuahua, chow, cocker spaniel, collie, corgi, dachshund, Dalmatian, Doberman, English setter, fox terrier, German shepherd, golden retriever, great dane, greyhound, griffon bruxellois, Irish setter, Irish wolfhound, King Charles spaniel, Labrador retriever, lhasa apso, mastiff, newfoundland, old English sheepdog, Papillion, Pekingese, pointer, Pomeranian, poodle, pug, Rottweiler, St. Bernard, saluki, Samoyed, schnauzer, Scottish terrier, Shetland sheepdog, shih tzu, Siberian husky, Skye terrier, springer spaniel, West Highland terrier, whip companion, Yorkshire terrier, etc.

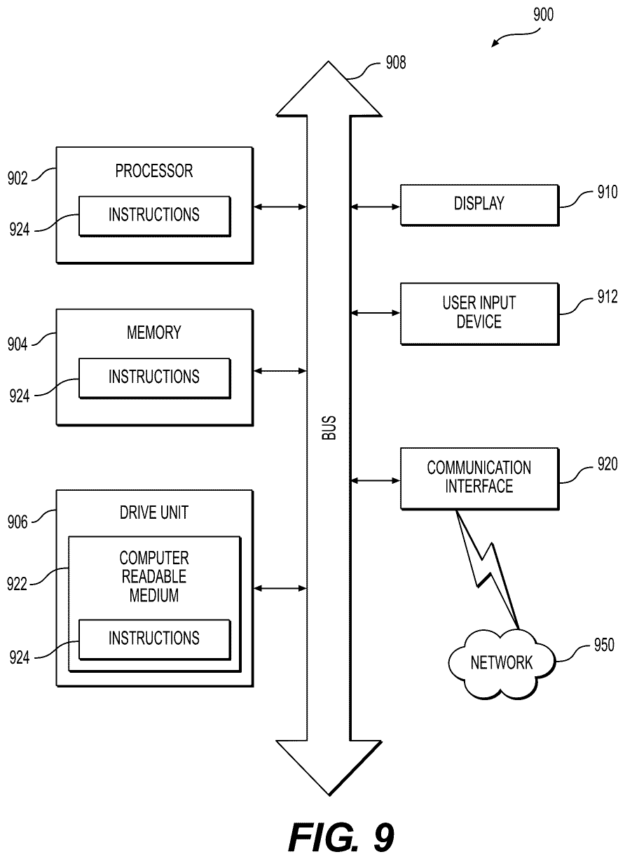

The terms “device” and “user device” may include, for example, without limitation, any electronic equipment, controlled by a central processing unit (CPU), for inputting information or data and displaying a user interface. A user device can send or receive signals, such as via a wired or wireless network, or can process or store signals, such as in memory as physical memory states. A device or user device as used in the present disclosure may include: a desktop computer; a mobile computer (e.g., a tablet computer, a laptop computer, or a netbook computer); a smartphone; a wearable computing device (e.g., smart watch); or the like, consistent with the computing device shown in FIG. 9.

As used herein, the term “pet metadata” may include, for example, without limitation, any one or combination of certain biological information or attributes of a pet including at least its breed, age, size, weight, body condition, shape of head (e.g., skull shape), predicted size category, predicted weight as an adult, and/or oral health data (e.g., oral microbiota data) related to common symptoms of gum or periodontal disease, such as the presence of bleeding gums, swollen (e.g., puffy) gums, tartar, change in eating habits, sensitive stomach, and/or halitosis, and/or medical history of gingivitis or periodontal disease. Pet metadata may also include information regarding a pet's oral care routine, such as the frequency of tooth brushing and/or the use of dental treats and chew toys. Additionally, for example, the pet metadata may include answers to questions pertaining to, but not limited to, the biological information and attributes discussed above.

The term “electronic medical records (EMRs)” refers to electronic records comprising pet medical history information. For the purposes of the current disclosure, EMRs may include pet metadata. For example, EMRs may include, but are not limited to, pet biological information or attributes of a pet including at least its breed, age, size, weight, body condition, shape of head (e.g., skull shape), predicted size category, predicted weight as an adult. EMRs may also include pet medical/health history information (e.g., symptoms, diagnoses, allergies, conditions, medications, lab/test results, and referral information etc.). For example, EMRs may include previous genetic (e.g., DNA) test results for a pet. The medical history may include a history of gingivitis or periodontal disease. Symptoms may include common symptoms of periodontal disease such as the presence of bleeding gums, swollen (e.g., puffy) gums, tartar, change in eating habits, sensitive stomach, and/or halitosis.

The term “oral sample” may include, for example, without limitation, plaque, saliva, or a sample of an oral fluid collected from the oral cavity of a pet.

The term “diagnosis” may include, for example, without limitation, the recognition and/or identification of a disease, the prediction of the course of a disease, as well as a conclusion with respect to a risk level associated with a disease (e.g., low risk, medium risk, high risk).

The terms “genetic test” and “genetic testing” may include, for example, without limitation, quantitative polymerase chain reaction (qPCR), loop-mediated isothermal amplification, high throughput sequencing, nanopore DNA sequencing, single-molecule real-time sequencing, next generation sequencing, Illumina sequencing, or other means for determining genetic sequences and/or genetic traits.

Certain non-limiting embodiments are described below with reference to block diagrams and operational illustrations of methods, processes, devices, and apparatus. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

In certain non-limiting embodiments, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors, such as an elastic computer cluster, and associated network and storage devices, as well as operating software and one or more database systems and application software that support the services provided by the server. The server, for example, can be a cloud-based server, a cloud-computing platform, or a virtual machine. Servers can vary widely in configuration or capabilities, but generally a server can include one or more central processing units and memory. A server can also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

For some non-limiting embodiments, a “network” should be understood to refer to a network that may couple devices so that communications can be exchanged, such as between a server and a user device or other types of devices, including between wireless devices coupled via a wireless network, for example. A network can also include mass storage, such as network attached storage (NAS), a storage area network (SAN), or other forms of computer or machine-readable media, for example. A network can include the Internet, one or more local area networks (LANs), one or more wide area networks (WANs), wire-line type connections, wireless type connections, cellular or any combination thereof. Likewise, sub-networks, which can employ differing architectures or can be compliant or compatible with differing protocols, can interoperate within a larger network. Various types of devices can, for example, be made available to provide an interoperable capability for differing architectures or protocols. As one illustrative example, a router can provide a link between otherwise separate and independent LANs.

Furthermore, devices or user devices, such as computing devices or other related electronic devices can be remotely coupled to a network, such as via a wired or wireless line or link, for example.

In certain non-limiting embodiments, a “wireless network” should be understood to couple user devices with a network. A wireless network can include virtually any type of wireless communication mechanism by which signals can be communicated between devices, between or within a network, or the like. A wireless network can employ standalone ad-hoc networks, mesh networks, wireless land area network (WLAN), cellular networks, or the like. A wireless network may be configured to include a system of terminals, gateways, routers, or the like coupled by wireless radio links, or the like, which can move freely, randomly, or organize themselves arbitrarily, such that network topology can change, at times even rapidly.

A wireless network can further employ a plurality of network access technologies, including Wi-Fi, Long Term Evolution (LTE), WLAN, Wireless Router (WR) mesh, or 2nd, 3rd, 4th, 5th generation (2G, 3G, 4G, or 5G) cellular technology, or the like. Network access technologies can allow wide area coverage for devices, such as user devices with varying degrees of mobility, for example.

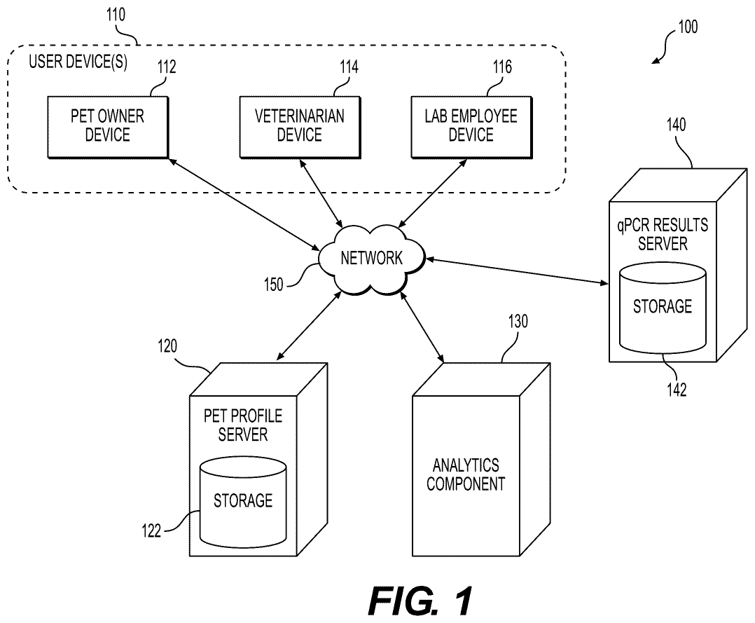

Referring now to the appended drawings, FIG. 1 shows an exemplary embodiment of a system 100 for processing data associated with a pet. In general, system 100 may comprise user device(s) 110, pet profile server 120, analytics component 130, qPCR results server 140, and network 150.

In system 100 of FIG. 1, user device(s) 110 may comprise one or more of pet owner device 112, veterinarian device 114, and lab employee device 116. The one or more user device(s) 110, such as pet owner device 112, veterinarian device 114, and/or lab employee device 116 may be used to input data, wherein the one or more user device(s) 110 may be a device consistent with the computing device depicted in FIG. 9, such as a desktop computer, a tablet, a laptop, a smart phone, a smart watch, etc.

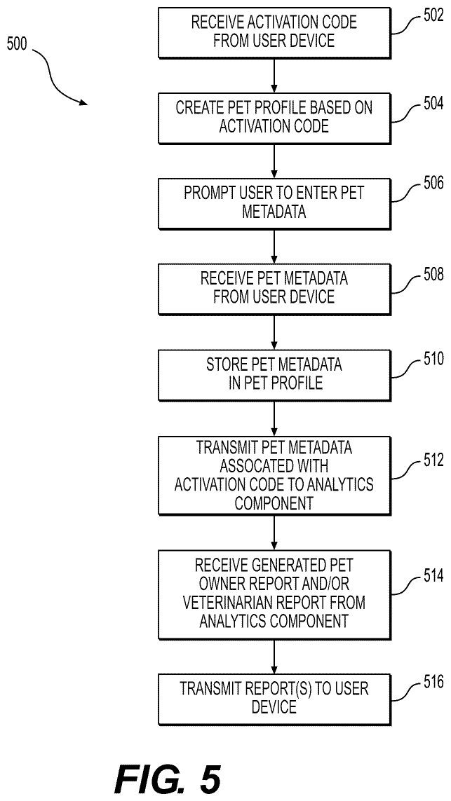

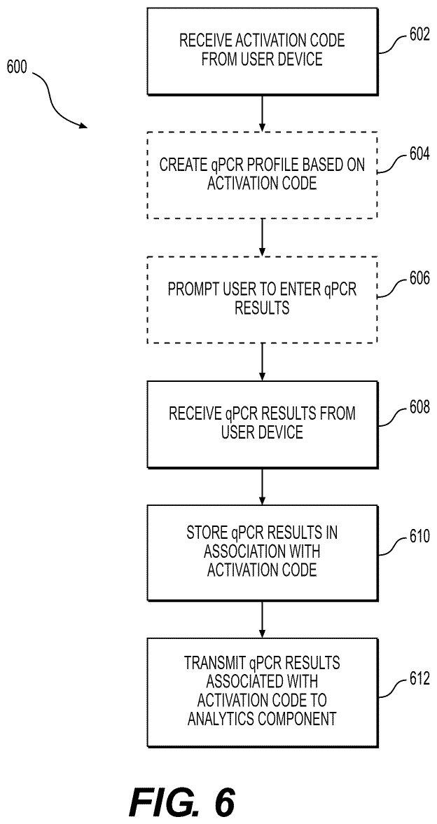

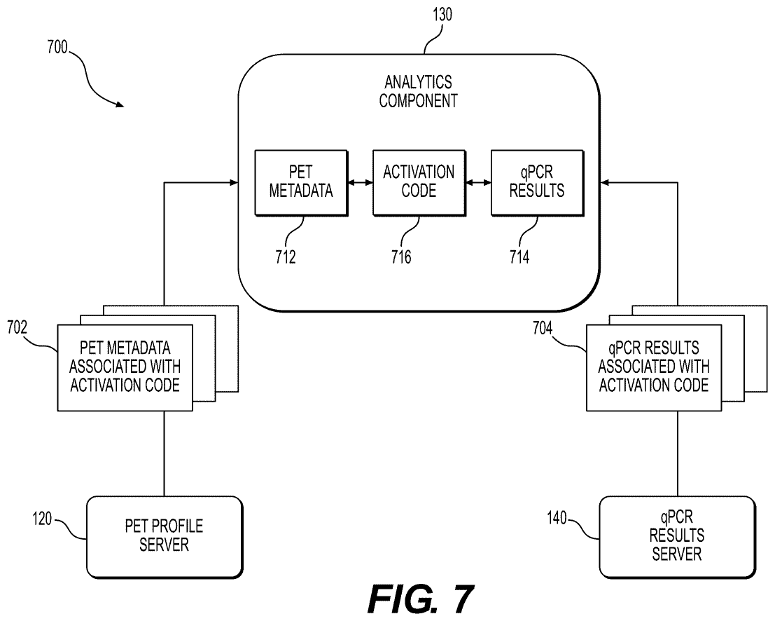

User device(s) 110 are coupled to network 150. Pet profile server 120, analytics component 130, and qPCR results server 140 are also each coupled to network 150. Network 150 connects each of user devices 110, pet profile server 120, analytics component 130, and qPCR results server 140, allowing for the transmission or sharing of data. Pet profile server 120 comprises storage 122. A more detailed description of pet profile server 120 and storage 122 is provided further below in reference to FIG. 5. qPCR results server 140 may comprise storage 142. A more detailed description of qPCR results server 140 and storage 142 is provided further below in reference to FIG. 6. Similarly, a more detailed description of analytics component 130 is provided further below in reference to FIG. 7.

It should be noted that, although system 100 depicts qPCR results server 140 for storing qPCR results, the system may comprise a server and storage for results obtained from any laboratory test conducted on an oral sample (e.g., plaque) collected from a pet. In some examples, a sequencing results server and storage may replace, or supplement, the qPCR results server 140 and storage 142 in system 100. The sequencing results may be obtained from high throughput sequencing, nanopore DNA sequencing, single-molecule real-time sequencing, next generation sequencing, Illumina sequencing, or other means for determining genetic sequences. In some embodiments, a server and storage may contain results from testing conducted on a biological sample collected from a pet, other than an oral sample, such as blood, stool, urine, hair, and tissue.

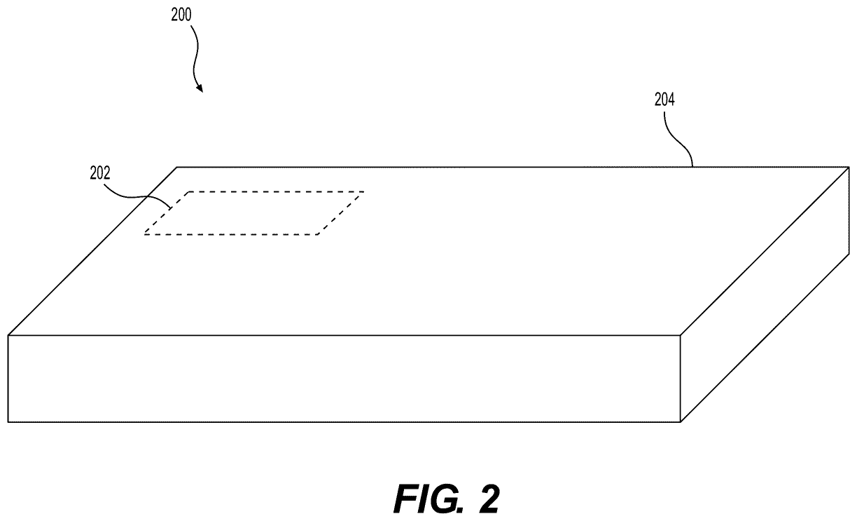

FIG. 2 illustrates an exemplary oral diagnostic kit 200. Oral diagnostic kit 200 comprises activation and identification component 202 and packaging 204. As depicted in FIG. 2, packaging 204 of oral diagnostic kit 200 may be in the shape of a rectangular box. Activation and identification component 202 is a label or label-like portion of oral diagnostic kit 200 that displays information that is unique to oral diagnostic kit 200 and that may be used to activate oral diagnostic kit 200. The placement of activation and identification component 202 is not limited to that explicitly shown in FIG. 2. Activation and identification component 202 may be placed at any suitable location on or within packaging 204. Activation and identification component 202 preferably contains an activation code representative of the sample being collected in the kit. Thus, the activation code may also be referred to as a sample identifier in the present disclosure.

Oral diagnostic kit 200 of FIG. 2 may be used in the context of system 100 of FIG. 1. For example, information, such as an activation code, displayed on activation and identification component 202 may be read by one or more users and input into user device(s) 110 of FIG. 1 to activate oral diagnostic kit 200. For instance, a user may input an activation code displayed on activation and identification component 202 into pet owner device 112 and/or veterinarian device 114 to activate oral diagnostic kit 200 and to create and/or populate a pet profile in system 100. Once oral diagnostic kit 200 is activated, the activation code displayed on activation and identification component 202 may be input into user device(s) 110 to access and/or view the pet profile associated with activated oral diagnostic kit 200 or to input data associated with the activation code such as pet metadata and/or qPCR results data for an oral sample collected with oral diagnostic kit 200.

Oral diagnostic kit 200 may contain an oral sample collection tool and/or container, as well as instructions. For example, oral diagnostic kit 200 may contain a swab for collecting the oral sample from the mouth of a pet and a container, preferably a dry container, for storing the collected oral sample. Alternatively, other suitable equipment for the collection of an oral sample may be included in oral diagnostic kit 200. Oral diagnostic kit 200 may also include instructions for using and/or activating the kit. The instructions may display each step in the process of using and activating the kit or the instructions may display a link to a website with detailed instructions. In some embodiments, the instructions may be included inside oral diagnostic kit 200. In other embodiments, the instructions may be printed and displayed at various locations on the outside of oral diagnostic kit 200.

While FIG. 2 depicts packaging 204 of oral diagnostic kit 200 in the shape of a rectangular box, oral diagnostic kit 200 may be in any suitable packaging shape. For example, packaging 204 may be in the form of an envelope or wallet, containing oral sample collection equipment and instructions. The packaging 204 of oral diagnostic kit 200 may be configured to securely enclose a container with a collected oral sample and to withstand shipping, handling, and/or delivery to a laboratory for testing.

Although FIG. 2 displays activation and identification component 202 in the upper left hand corner of oral diagnostic kit 200, the activation and identification component 202 may be displayed anywhere on or within oral diagnostic kit 200. In some embodiments, activation and identification component 202 may be a detachable label. In other embodiments, activation and identification component 202 may be printed directly on packaging 204. Further, in some examples, activation and identification component 202 may be displayed through a window or opening in packaging 204. In other embodiments, activation identification component 202 may not be visible from packaging 204, and may be placed on an inner surface or inside of packaging 204. Activation and identification component 202, preferably displays an activation code. In some embodiments, activation and identification component 202 may display a quick response (QR) code, a bar code, or another user device readable code.

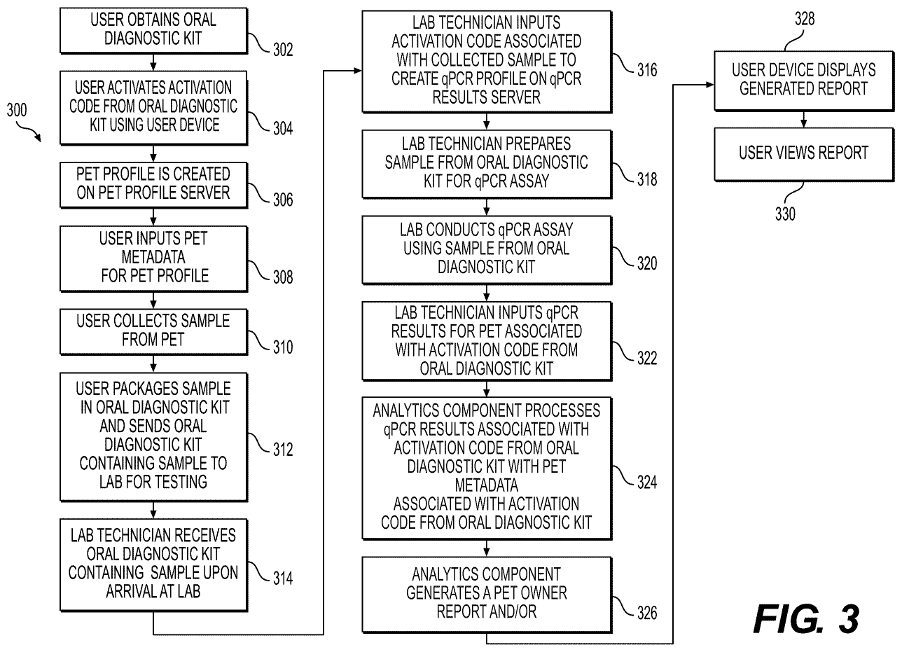

FIG. 3 depicts a flowchart illustrating exemplary process 300 performed by system 100 for generating user reports based on pet metadata and qPCR results obtained from disparate sources. In step 302, a user obtains oral diagnostic kit 200. In some embodiments, a user may be a pet owner. In other embodiments, a user may be a veterinarian. Oral diagnostic kit 200 may be purchased by a pet owner from a pet store or another store that sells pet products, or as a part of a medical service provided by a veterinary clinic. A pet owner may also have the option to purchase oral diagnostic kit 200 online. Oral diagnostic kit 200 may then be shipped to a location specified by a pet owner, such as a pet owner's address. In certain examples, a pet owner may obtain oral diagnostic kit 200 from a veterinarian. A veterinarian may order oral diagnostic kit 200 in bulk. Oral diagnostic kit 200 may be stored in a veterinarian facility for later use by a veterinarian or the veterinarian may distribute oral diagnostic kit 200 to a pet owner upon request.

Upon obtaining oral diagnostic kit 200 in step 302, in step 304, a user locates activation and identification component 202 on packaging 204 of oral diagnostic kit 200. Instructions included with oral diagnostic kit 200 will provide information regarding where the user can input activation and identification component 202 information, such as an activation code. For example, a user may be directed to a user interface for a mobile application or a website. The user may then input the information displayed on activation and identification component 202, such as an activation code, via a user interface rendered on a user device 110. By inputting the activation code from oral diagnostic kit 200 in step 304, the user activates the kit. Alternatively, in embodiments where activation and identification component 202 displays a QR code (or any other user device readable code), user device 110 may be used to scan the code. In embodiments where the user is a pet owner, the pet owner activates oral diagnostic kit 200 on pet owner device 112. In embodiments where the user is a veterinarian, the veterinarian activates oral diagnostic kit 200 on veterinarian device 114.

In step 306, a pet profile is created on pet profile server 120, after the oral diagnostic kit is activated. A user interface enabled by the pet profile server 120, via a webpage or an app, may display a message relaying that the oral diagnostic kit, oral diagnostic kit 200, has been activated and that a pet profile has been created. The pet profile that has been created may be displayed on a user device 110, such as pet owner device 112 or veterinarian device 114, for either the pet owner or the veterinarian. The user may be able to access and/or view the pet profile at any time after the activation of oral diagnostic kit 200. The pet profile may be associated with the activation code used to activate oral diagnostic kit 200.

In step 308, the user is prompted to input pet metadata via pet owner device 112 and/or veterinarian device 114. The pet metadata may be stored in storage 122 and may be associated with the activation code used to activate oral diagnostic kit 200. In other words, the pet metadata may be stored in association with the activation code, or may be stored in association with the pet profile created for oral diagnostic kit 200. The pet metadata that is input by a user, such as the pet owner and/or the veterinarian, and stored in storage 122 may include but is not limited to its breed, age, size, weight, and answers to questions regarding a pet's oral health, such as the current oral care routine and/or current signs or symptoms of periodontal disease.

In some embodiments, a first user who activates oral diagnostic kit 200 and inputs pet metadata may have the option of inputting the contact information of a user other than the first user in step 308. The contact information of the user other than the first user may be stored in storage 122 on pet profile server 120 in system 100, in association with the pet profile associated with the activated oral diagnostic kit 200. In other words, the contact information of a user other than the first user may be associated with the activation code from oral diagnostic kit 200 on pet profile server 120. In these embodiments, a user other than the first user may also have access to the pet profile on pet profile server 120. For example, when the first user is a pet owner, after activating oral diagnostic kit 200 and inputting pet metadata for the pet profile, the pet owner has the option to enter the contact information of the pet's veterinarian. As a result, the veterinarian also has access to the pet profile on pet profile server 120 and may input additional pet metadata to supplement the pet metadata already input by the pet owner.

Alternatively, when the first user is a veterinarian, after activating oral diagnostic kit 200 and inputting pet metadata for the pet profile, the veterinarian has the option to enter the contact information of the pet owner. As a result, the pet owner also has access to the pet profile on pet profile server 120 and may input additional pet metadata to supplement the pet metadata already input by the veterinarian.

In other embodiments, during step 308, all of the pet metadata is input by only one user. This user may be either the pet owner or the veterinarian. In preferred embodiments, the user is the pet owner. In step 308, the user still has the option to enter the contact information of another user, which may be stored in storage 122 on pet profile server 120 in association with the pet profile for activated oral diagnostic kit 200. In these embodiments, both the user who input the pet metadata and the user whose contact information is stored on pet profile server 120 may have access to viewing the pet profile on pet profile server 120 and may subsequently receive a report generated by analytics component 130.

Although FIG. 3 depicts a user inputting pet metadata for the pet profile in step 308, in some examples herein, the pet profile server 120 may receive pet metadata associated with the pet from an electronic medical records (EMR) database. For example, step 308 may include receiving pet metadata from electronic medical records (EMRs) for the pet in addition to or as an alternative to receiving pet metadata input by a user. In these examples, when the pet profile is created for the pet, pet metadata from EMRs for the pet is transmitted to the pet profile server 120. In at least one example, data associated with results from previous genetic tests performed in association with the pet may be linked to the EMR. For example, a pet EMR may contain data associated with previous genetic test results, which may be transmitted to the pet profile server 120.

In step 310, the user who obtained oral diagnostic kit 200 may collect an oral sample from a pet using oral diagnostic kit 200. Oral diagnostic kit 200 may contain instructions pertaining to use of the oral diagnostic kit. The user may collect an oral sample (e.g., plaque) by using an oral sample collection tool, such as a handle with a brush on the end or a swab. The user may swab the mouth of the pet by brushing or swabbing at least one of the gums, the gum-line, the teeth, the tongue, the cheek, and/or the roof of the mouth with the oral sample collection tool. Once the oral sample has been collected, the oral sample collection tool containing the sample may be placed in a container. Alternatively, other suitable methods of collecting an oral sample may be utilized. For example, a pet may be provided a chew toy to stimulate salivation, and the saliva may be collected and stored in a container from oral diagnostic kit 200.

Once the oral sample has been collected and placed in a container, in step 312, the user packages the oral sample in oral diagnostic kit 200. In some embodiments, the oral sample may be repackaged and sealed in oral diagnostic kit 200. After packaging the oral sample, the user may ship the oral diagnostic kit containing the oral sample to the laboratory or testing facility specified on oral diagnostic kit 200. In certain embodiments, the user may have the option to drop the oral diagnostic kit containing the oral sample off at a designated lab specimen drop box.

In step 314, the oral sample arrives at a laboratory or testing facility and a lab employee, such as a lab technician, receives the oral diagnostic kit containing the oral sample. In step 316, the lab technician inputs the activation code associated with the collected sample on lab employee device 116. For example, a lab technician locates activation and identification component 202 and inputs the activation code displayed thereon on a user interface displayed on lab employee device 116. Upon inputting the activation code, a test results profile associated with the activation code for oral diagnostic kit 200 will be created on a test results server. In some embodiments, the test results profile will be a qPCR results profile and the test results server will be qPCR results server 140. The lab technician may access the qPCR results profile using lab employee device 116.

In step 318, the lab technician unpacks the sample from oral diagnostic kit 200 and prepares the sample for a qPCR assay. As an example, a quantitative polymerase chain reaction (qPCR) assay is performed to measure an amount of a microbial nucleic acid in the sample and to detect the presence of bacteria or other markers associated with an oral disease or good oral health. In step 320, the lab conducts a qPCR assay on the oral sample. In some examples, a universal qPCR assay (UniB) which detects the presence of bacterial DNA in a pet's oral microbiome may be conducted. For example, bacterial species of interest in the oral sample may be determined and measured, followed by calculating the abundance of the species of interest relative to all of the bacteria present in the oral sample. Alternatively, other tests (e.g., genetic tests) or assays may be performed on the sample for the purposes of identifying markers associated with oral disease or other diseases of interest.

After conducting the qPCR assay, in step 322, a lab technician inputs the qPCR results via a user interface of lab employee device 116. The qPCR results may be stored in qPCR results server 140. The lab technician may access the qPCR results profile previously created in step 316. The qPCR results entered may be associated with the activation code from oral diagnostic kit 200, or may be stored within, or in association with, the qPCR results profile created for oral diagnostic kit 200. The qPCR results may be stored in storage 142 on qPCR results server 140.

In step 324, analytics component 130 processes qPCR results associated with the activation code from oral diagnostic kit 200 with pet metadata (e.g., pet breed, age, size etc.) associated with the activation code from oral diagnostic kit 200. For example, analytics component 130 may retrieve pet metadata associated with the activation code from pet profile storage 122 on pet profile server 120, as well as qPCR results associated with the activation code from qPCR results storage 142 on qPCR results server 140. The pet metadata may be processed with the qPCR results, once analytics component 130 determines a correlation between the pet metadata and the qPCR results based on the activation code from oral diagnostic kit 200.

After processing step 324, in step 326, analytics component 130 generates at least one diagnosis and/or at least one health recommendation based on the processing, in the form of one or more reports. For example, a report for the pet owner may be generated or a report for the veterinarian may be generated. In some embodiments, reports for both the pet owner and the veterinarian may be generated by analytics component 130.

In step 328, a user device 110 displays the generated report containing the at least one diagnosis and/or at least one health recommendation. For example, pet owner device 112 may display a report that has been generated specifically for the pet owner. Veterinarian device 114 may display a report that has been generated specifically for the veterinarian. In step 330, the pet owner and/or the veterinarian may view their respective reports on their respective user devices 110.

Although FIG. 3 depicts steps 312 through 322 in which a user, such as a pet owner or a veterinarian, sends the oral sample collected to a lab for testing, wherein the lab conducts the testing (e.g., qPCR assay) and inputs the test results, in some embodiments herein, the testing of the oral sample may be performed in real-time. In these embodiments, the oral sample is not sent to a lab and steps 312 through 322 are skipped. For example, the testing/assay is performed at the time the oral sample is collected and the results are generated. A readout of the results (e.g., qPCR results) may be supplied directly to the user (e.g., pet owner or veterinarian). The results may be manually input by the pet owner or veterinarian via a user interface of pet owner device 112 or veterinarian device 114. In other examples, a QR code or another user device readable code associated with the test results may be generated. Pet owner device 112 or veterinarian device 114 may scan or photograph the code and the process of 300 may proceed to step 324. Such steps allow for the elimination of outside lab testing and associated wait times.

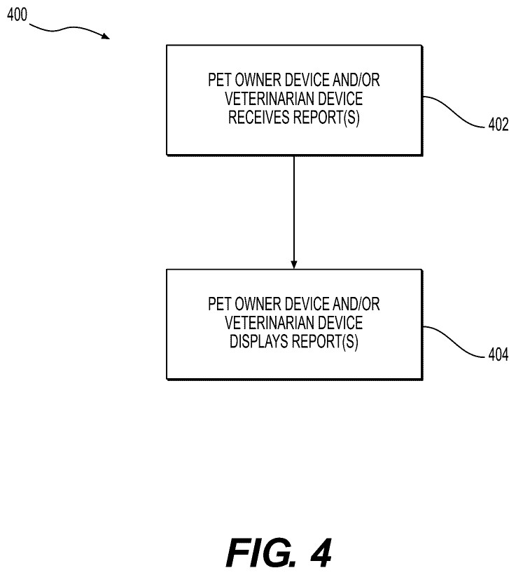

FIG. 4 illustrates a method 400 of receiving and displaying a report on a user device 110 in system 100. In step 402, pet owner device 112 and/or veterinarian device 114 receives a report generated by analytics component 130. In one embodiment, the pet owner device 112 and/or veterinarian device 114 receives a report generated for each respective user from pet profile sever 120. In some embodiments, pet profile server 120 transmits the report(s) directly to pet owner device 112 and/or veterinarian device 114, while in some embodiments pet profile server 120 transmits a message to pet owner device 112 and/or veterinarian device 114 to view the report(s) on pet profile server 120, or download the report(s) from pet profile server 120. The report(s) may be viewed on or downloaded from pet profile server 120 at any time after the report(s) have been generated. In other embodiments, pet owner device 112 and/or veterinarian device 114 may receive the report(s) directly from analytics component 130. Further, in other embodiments, pet owner device 112 may receive a report from veterinarian device 114 upon the report being transmitted or downloaded to veterinarian device 114, or veterinarian device 114 may receive a report from pet owner device 112 upon the report being transmitted or downloaded to pet owner device 112. In step 404, the pet owner device 112 and/or veterinarian device 114 displays the report(s).

FIG. 5 depicts a flowchart illustrating exemplary process 500 performed by pet profile server 120 in system 100 of FIG. 1. In step 502, pet profile server 120 receives an activation code associated with oral diagnostic kit 200 from one of user device(s) 110, such as pet owner device 112 or veterinarian device 114. Upon receiving the activation code, in step 504, pet profile server creates a pet profile based on the activation code from step 502 on pet profile server 120. The pet profile may be associated with or identified by the activation code. Once the pet profile has been created, a user interface on a user device 110 may be configured to display the pet profile. In some embodiments, creation of a pet profile may not be necessary. For instance, instead of creating a pet profile, pet profile server 120 may merely store the activation code received from a user in storage 122, and may store any subsequently-received information from the user (e.g., pet metadata) in association with the activation code.

In step 506, pet profile server 120 prompts a user, such as a pet owner or a veterinarian, to input pet metadata for the pet profile via pet owner device 112 or veterinarian device 114. The user may be prompted to answer a questionnaire containing specific questions pertaining to the desired pet metadata. An exemplary questionnaire may inquire about the pet's name, owner contact information, veterinarian contact information, as well as the pet's age, breed, size, weight, body condition, shape of head, predicted size category, and predicted weight as an adult. Other pet metadata that may be inquired about the pet includes the presence of common symptoms of oral health disease, such as bleeding gums, swollen gums, tartar, change in eating habits, sensitive stomach, and/or halitosis. Pet owners may also be asked about the pet's current oral care routine, such as the frequency of tooth brushing and the types of dental treats used. Veterinarians may be asked about the medical history of the pet, specifically with respect to gingivitis and periodontal disease.

Pet profile server 120 may prompt the user to enter the pet metadata manually, such as by typing responses to each of the questions, or to select the answers from a dropdown menu. Alternatively, oral diagnostic kit 200 may contain a list of questions pertaining to the requisite pet metadata and pet profile server 120 may prompt the user to enter the pet metadata via a user interface rendered on one of user device(s) 110.

In some examples, pet profile server 120 may prompt the user to answer whether they are the pet owner or the veterinarian. After answering this question, when the user is a pet owner, the pet owner may be prompted to enter the contact information, such as an email address, of the pet's veterinarian. If the pet does not have a veterinarian, the pet owner may have the option of having a local veterinarian recommended based on their home address. When the user is a veterinarian, the veterinarian may be prompted to enter an email address of the pet owner. In certain embodiments, once the user has input pet metadata, pet profile server 120 may send an email to another user, such as the pet owner or the veterinarian depending on the initial user, providing a link to the pet profile created. Pet profile server 120 may then prompt the other user to input additional pet metadata to supplement the pet metadata previously entered by the initial user.

In step 508, pet profile server 120 receives pet metadata from one or more of user device(s) 110, such as pet owner device 112 and/or veterinarian device 114. After receiving pet metadata, pet profile server 120 stores the pet metadata in storage 122 in step 510. In some embodiments, storage 122 may be a database. In other embodiments, storage 122 may be a file system storage. The pet metadata may be stored within, or in association with, the pet profile created for oral diagnostic kit 200 in pet profile server 120. In some embodiments, the pet metadata may merely be associated with the activation code for oral diagnostic kit 200 in pet profile server 120, irrespective of the presence of a corresponding pet profile.

Although FIG. 5 depicts prompting a user to enter pet metadata and receiving pet metadata from a user device in steps 506 and 508, respectively, in some examples herein, pet metadata associated with the pet may be received from an electronic medical records (EMR) database. In these examples, after the pet profile is created, pet metadata from EMRs stored in an EMR database is received from the EMR database. In some examples, the pet profile server 120 may receive pet metadata from electronic medical records (EMRs) for the pet in addition to or as an alternative to receiving pet metadata input by a user. In at least one example, in step 506 a user (e.g., pet owner) may only be prompted to enter the pet's current oral care routine and user contact information via a user device. All other pet metadata associated with the pet may be received from an EMR database and subsequently stored in the pet profile in step 510. In another example, steps 506 and 508 may be skipped and all pet metadata associated with the pet may be received from an EMR database. The pet metadata received is subsequently stored in the pet profile in step 510.

In step 512, pet profile server 120 transmits the pet metadata associated with the activation code of oral diagnostic kit 200 to analytics component 130. In this step, both the pet metadata and the activation code may be transmitted to analytics component 130. To that end, a pet profile that contains the pet metadata and is associated with the activation code may be transmitted to analytics component 130. Alternatively, in embodiments where a pet profile was never created, the pet metadata may be transmitted to analytics component 130 along with the corresponding activation code.

In step 514, pet profile server 120 receives one or more reports generated by analytics component 130. These reports include a pet owner report and a veterinarian report. The reports may be stored in storage 122. In step 516, pet profile server 120 transmits the one or more reports, such as a pet owner report and a veterinarian report, to one or more user device(s) 110. For example, pet profile server 120 may transmit a pet owner report to pet owner device 112 and a veterinarian report to veterinarian device 114. In some embodiments, pet profile server 120 may only transmit one report to one user device 110. In other embodiments, pet profile server 120 may transmit both a pet owner report and a veterinarian report to pet owner device 112 or pet profile server 120 may transmit both a pet owner report and a veterinarian report to veterinarian device 114. As discussed above in reference to FIG. 3, the reports may alternatively be transmitted from analytics component 130 directly to user device(s) 110.

FIG. 6 depicts a flowchart illustrating exemplary process 600 performed by qPCR results server 140 in system 100 of FIG. 1. In step 602, qPCR results server 140 receives an activation code associated with oral diagnostic kit 200 from a user device 110, such as lab employee device 116. In process 600, the preferred user is a lab technician and the preferred user device is lab employee device 116. However, in some embodiments, a lab employee device 116 may transmit the qPCR results to another user device 110, such as veterinarian device 114. In these embodiments, a veterinarian could use veterinarian device 114 to input the activation code associated with oral diagnostic kit 200 and subsequently input the qPCR results onto qPCR results server 140.

Following step 602, an optional step 604 may be performed by qPCR results server 140, wherein qPCR results server 140 creates a qPCR profile based on the activation code. Following optional step 604, optional step 606 may be performed by qPCR results server 140, wherein qPCR results server 140 prompts a user, such as a lab technician, to input qPCR results on qPCR results server 140 via lab employee device 116. In some embodiments, steps 604 and 606 may be performed, while in other embodiments, step 604 and/or step 606 may be skipped. For example, in some embodiments, a lab technician may input qPCR results after inputting the activation code from oral diagnostic kit 200 on lab employee device 116, without any prompt being displayed to the lab technician for qPCR results. Therefore, in these embodiments, qPCR results server would skip steps 604 and 606, and associate the received activation code with the subsequently-received qPCR results.

In step 608, qPCR results server 140 receives qPCR results from a user device 110, such as lab employee device 116. In step 610, qPCR results server 140 stores the qPCR results in association with the activation code from oral diagnostic kit 200 in storage 142. In some embodiments, storage 142 may be a database. In other embodiments, storage 142 may be a file system storage. In step 612, qPCR results server 140 transmits the qPCR results associated with the activation code from oral diagnostics kit 200 to analytics component 130.

FIG. 7 depicts a block diagram illustrating a linkage between pet metadata and qPCR results received from pet profile server 120 and qPCR results server 140 respectively. As discussed with respect to FIG. 5 above, pet profile server 120 stores pet metadata associated with the activation code from oral diagnostic kit 200. Once pet profile server 120 creates a pet profile based on an activation code from oral diagnostic kit 200 and receives pet metadata via user device(s) 110, the pet metadata entered or received from an EMR database becomes associated with the activation code, and is stored in pet profile server 120 as pet metadata associated with activation code 702. Pet profile server 120 may then transmit pet metadata associated with activation code 702 to analytics component 130.

As discussed with respect to FIG. 6 above, qPCR results server 140 stores qPCR results associated with the activation code from oral diagnostic kit 200. Once qPCR results server 140 receives the activation code from oral diagnostic kit 200 and receives qPCR results via user device 110, such as lab employee device 116, the qPCR results entered become associated with the activation code, and are stored in qPCR results server 140 as qPCR results associated with activation code 704. qPCR server 140 may then transmit qPCR results associated with activation code 704 to analytics component 130.

Once pet metadata associated with activation code 702 and qPCR results associated with activation code 704 are received by analytics component 130, analytics component 130 determines a correlation between the pet metadata and the qPCR results based on the activation code. For example, one or more processors of analytics component 130 retrieves activation code 716 from the received information, and establishes a linkage or correlation between different types of data that are each associated with the activation code 716. Analytics component 130 then retrieves pet metadata 712 and qPCR results 714 that are each associated with activation code 716 for further processing.

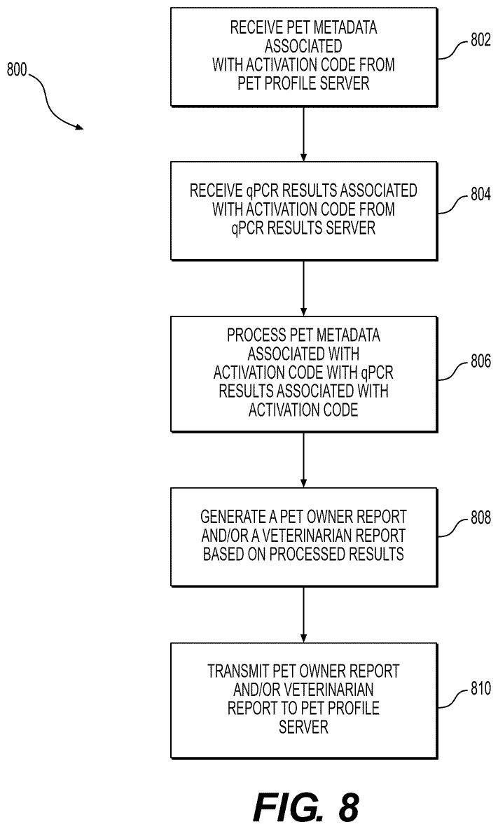

FIG. 8 depicts a flow chart illustrating exemplary process 800 performed by analytics component 130 in system 100 of FIG. 1. As discussed above with respect to FIG. 7, in step 802, analytics component 130 receives pet metadata associated with the activation code from oral diagnostic kit 200 from pet profile server 120. In step 804, analytics component 130 receives qPCR results associated with the activation code from oral diagnostic kit 200 from qPCR results server 140. In step 806, analytics component 130 processes pet metadata associated with the activation code from oral diagnostic kit 200 with qPCR results associated with the activation code from oral diagnostic kit 200. As explained with respect to FIG. 7, one or more processors of analytics component 130 are configured to determine a correlation between the pet metadata and the qPCR results based on the activation code, prior to processing.

After the processing of step 806, in step 808, analytics component 130 generates at least one diagnosis and/or at least one health recommendation based on the processed results from step 806. The at least one diagnosis and/or at least one health recommendation may be generated as one or more reports for a pet owner and/or a veterinarian. In step 810, analytics component 130 transmits the one or more reports, such as a pet owner report and/or a veterinarian report to pet profile server 120. In some embodiments, analytics component 130 may transmit the one or more reports directly to user device(s) 110. For example, analytics component 130 may transmit a pet owner report to pet owner device 112 and analytics component 130 may transmit a veterinarian report to veterinarian device 114.

Analytics component 130 may process the pet metadata associated with the activation code from oral diagnostic kit 200 with the qPCR results associated with the activation code from oral diagnostic kit 200 after determining a correlation based on the activation code, to provide at least one diagnosis. The processed results may also be used to provide at least one health recommendation, with respect to aftercare treatment and/or an oral care routine. An exemplary report containing at least one diagnosis may explain that a pet is at either high, medium, or low risk for having gum disease based on the processed qPCR and pet metadata results. When the pet is at high or medium risk for having gum or periodontal disease, the diagnosis report may also provide additional information predicting the progression of the disease. In some embodiments where a veterinarian report is generated, the veterinarian report may only contain information pertaining to the diagnosis. In other embodiments, the veterinarian report may also contain a recommended treatment plan for the veterinarian, in addition to a diagnosis.

An exemplary report containing at least one health recommendation may provide a recommended oral care routine or plan, based on the processed qPCR and pet metadata results. For example, the report may recommend increased tooth brushing, use of dental treats/chews, oral rinses/gels, and water additives. The report may also provide a recommended diet for the pet and a recommended date for scheduling the next visit to the veterinarian. In cases where the pet is diagnosed as having a high risk for periodontal disease, a report may recommend that the pet visit the veterinarian immediately. In instances where the risk level is lower, the recommendation may be to re-test with the oral diagnostic kit after one year to monitor changes. In other instances where the risk level is low, rewards may be recommended.

In some embodiments, reports containing the at least one health recommendation may only be generated for the pet owner as a pet owner report. The pet owner report may also recommend certain dental treats and advise on suitable chew toys for pets. In some examples, the pet owner report may even provide a hyperlink that connects the pet owner to recommended oral care products that can be purchased for their pets. The pet owner report may also be linked to a tooth brushing guide and to an application that provides daily oral care reminders. In some examples, the pet owner report may also include a “Call my veterinarian” button for contacting their veterinarian. In certain embodiments, the pet owner report and/or the veterinarian report may include at least one diagnosis and at least one health recommendation.

It should be understood that embodiments in this disclosure are exemplary only, and that other embodiments may include various combinations of features from other embodiments, as well as additional or fewer features. For example, while some of the embodiments above pertain to using an oral diagnostic kit to assess pet oral health and to detect oral health disease, the oral diagnostic kit may also be used to assess the overall health of pets and to detect other common pet diseases. Further, while some of the embodiments above pertain to qPCR assays, any suitable assays for detecting bacteria associated with an oral disease or good oral health may be used.

FIG. 9 illustrates an implementation of a computer system that may execute techniques presented herein. The computer system 900 can include a set of instructions that can be executed to cause the computer system 900 to perform any one or more of the methods or computer based functions disclosed herein. The computer system 900 may operate as a standalone device or may be connected, e.g., using a network, to other computer systems or peripheral devices.

In a networked deployment, the computer system 900 may operate in the capacity of a server or as a client user computer in a server-client user network environment, or as a peer computer system in a peer-to-peer (or distributed) network environment. The computer system 900 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 900 can be implemented using electronic devices that provide voice, video, or data communication. Further, while a single computer system 900 is illustrated, the term “system” shall also be taken to include any collection of systems or sub-systems that individually or jointly execute a set, or multiple sets, of instructions to perform one or more computer functions.

As illustrated in FIG. 9, the computer system 900 may include a processor 902, e.g., a central processing unit (CPU), a graphics processing unit (GPU), or both. The processor 902 may be a component in a variety of systems. For example, the processor 902 may be part of a standard personal computer or a workstation. The processor 902 may be one or more general processors, digital signal processors, application specific integrated circuits, field programmable gate arrays, servers, networks, digital circuits, analog circuits, combinations thereof, or other now known or later developed devices for analyzing and processing data. The processor 902 may implement a software program, such as code generated manually (i.e., programmed).

The computer system 900 may include a memory 904 that can communicate via a bus 908. The memory 904 may be a main memory, a static memory, or a dynamic memory. The memory 904 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 904 includes a cache or random-access memory for the processor 902. In alternative implementations, the memory 904 is separate from the processor 902, such as a cache memory of a processor, the system memory, or other memory. The memory 904 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 904 is operable to store instructions executable by the processor 902. The functions, acts or tasks illustrated in the figures or described herein may be performed by the programmed processor 902 executing the instructions stored in the memory 904. The functions, acts or tasks are independent of the particular type of instructions set, storage media, processor or processing strategy and may be performed by software, hardware, integrated circuits, firm-ware, micro-code and the like, operating alone or in combination. Likewise, processing strategies may include multiprocessing, multitasking, parallel processing and the like.

As shown, the computer system 900 may further include a display unit 910, such as a liquid crystal display (LCD), an organic light emitting diode (OLED), a flat panel display, a solid-state display, a cathode ray tube (CRT), a projector, a printer or other now known or later developed display device for outputting determined information. The display 910 may act as an interface for the user to see the functioning of the processor 902, or specifically as an interface with the software stored in the memory 904 or in the drive unit 906.

Additionally or alternatively, the computer system 900 may include an input device 912 configured to allow a user to interact with any of the components of system 900. The input device 912 may be a number pad, a keyboard, or a cursor control device, such as a mouse, or a joystick, touch screen display, remote control, or any other device operative to interact with the computer system 900.

The computer system 900 may also or alternatively include a disk or optical drive unit 906. The disk drive unit 906 may include a computer-readable medium 922 in which one or more sets of instructions 924, e.g. software, can be embedded. Further, the instructions 924 may embody one or more of the methods or logic as described herein. The instructions 924 may reside completely or partially within the memory 904 and/or within the processor 902 during execution by the computer system 900. The memory 904 and the processor 902 also may include computer-readable media as discussed above.

In some systems, a computer-readable medium 922 includes instructions 924 or receives and executes instructions 924 responsive to a propagated signal so that a device connected to a network 950 can communicate voice, video, audio, images, or any other data over the network 950. Further, the instructions 924 may be transmitted or received over the network 950 via a communication port or interface 920, and/or using a bus 908. The communication port or interface 920 may be a part of the processor 902 or may be a separate component. The communication port 920 may be created in software or may be a physical connection in hardware. The communication port 920 may be configured to connect with a network 950, external media, the display 910, or any other components in system 900, or combinations thereof. The connection with the network 950 may be a physical connection, such as a wired Ethernet connection or may be established wirelessly as discussed below. Likewise, the additional connections with other components of the system 900 may be physical connections or may be established wirelessly. The network 950 may alternatively be directly connected to the bus 908.

While the computer-readable medium 922 is shown to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that cause a computer system to perform any one or more of the methods or operations disclosed herein. The computer-readable medium 922 may be non-transitory, and may be tangible.

The computer-readable medium 922 can include a solid-state memory such as a memory card or other package that houses one or more non-volatile read-only memories. The computer-readable medium 922 can be a random-access memory or other volatile re-writable memory. Additionally or alternatively, the computer-readable medium 922 can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that may include the apparatus and systems of various implementations can broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The computer system 900 may be connected to one or more networks 950. The network 950 may define one or more networks including wired or wireless networks. The wireless network may be a cellular telephone network, an 802.11, 802.16, 802.20, or WiMax network. Further, such networks may include a public network, such as the Internet, a private network, such as an intranet, or combinations thereof, and may utilize a variety of networking protocols now available or later developed including, but not limited to TCP/IP based networking protocols. The network 950 may include wide area networks (WAN), such as the Internet, local area networks (LAN), campus area networks, metropolitan area networks, a direct connection such as through a Universal Serial Bus (USB) port, or any other networks that may allow for data communication. The network 950 may be configured to couple one computing device to another computing device to enable communication of data between the devices. The network 950 may generally be enabled to employ any form of machine-readable media for communicating information from one device to another. The network 950 may include communication methods by which information may travel between computing devices. The network 950 may be divided into sub-networks. The sub-networks may allow access to all of the other components connected thereto or the sub-networks may restrict access between the components. The network 950 may be regarded as a public or private network connection and may include, for example, a virtual private network or an encryption or other security mechanism employed over the public Internet, or the like.

In accordance with various implementations of the present disclosure, the methods described herein may be implemented by software programs executable by a computer system. Further, in an exemplary, non-limited implementation, implementations can include distributed processing, component/object distributed processing, and parallel processing. Alternatively, virtual computer system processing can be constructed to implement one or more of the methods or functionality as described herein.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosed embodiments are not limited to any particular implementation or programming technique and that the disclosed embodiments may be implemented using any appropriate techniques for implementing the functionality described herein. The disclosed embodiments are not limited to any particular programming language or operating system.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A system for providing pet health diagnostic and aftercare information, comprising:
a first user device;
a second user device;
a pet profile server comprising a pet profile storage and configured to:
receive an activation code from the first user device;
receive pet metadata from the first user device; and
store the pet metadata in association with the activation code in the pet profile storage;
a test results server comprising a test results storage and configured to:
receive the activation code from the second user device;
receive test results data from the second user device; and
store the test results data in association with the activation code in the test results storage; and
an analytics component executed by one or more processors and configured to:
receive the pet metadata associated with the activation code from the pet profile server;
receive the test results data associated with the activation code from the test results server;
upon determining a correlation between the pet metadata and the test results data based on the activation code, process the pet metadata with the test results data;
generate at least one diagnosis and/or at least one health recommendation based on the processed data and
generate one or more reports based on the at least one diagnosis and/or the at least one health recommendation.

2. The system of claim 1, wherein the pet profile storage is a pet profile database.

3. The system of claim 1, wherein the test results storage is a test results database.

4. The system of claim 1, wherein the activation code is received from the first user device upon a first user identifying and providing the activation code from an oral diagnostic kit.

5. The system of claim 4, wherein the activation code is received from the second user device upon a second user identifying and providing the activation code from the oral diagnostic kit.

6. The system of claim 1, wherein the test results data comprises test results from a quantitative polymerase chain reaction (qPCR) assay.

7. The system of claim 1, wherein the analytics components is further configured to transmit the one or more reports to the pet profile server.

8. The system of claim 1, wherein the one or more reports comprise a pet owner report and/or a veterinarian report.

9. The system of claim 1, wherein the first user device is configured to:
receive, from a first user via a user interface, the activation code identified from an oral diagnostic kit;
transmit the activation code to the pet profile server;
receive, from the first user via the user interface, the pet metadata after receiving the activation code;
transmit the pet metadata to the pet profile server; and
receive the one or more generated reports.

10. The system of claim 9, wherein the first user device is further configured to display the one or more generated reports via the user interface.

11. The system of claim 9, wherein the first user device is further configured to receive, from the first user via the user interface, contact information.

12. The system of claim 11, wherein the contact information is associated with a third user.

13. The system of claim 12, wherein the first user is one of a pet owner or a veterinarian, and the third user is the other of the pet owner or the veterinarian.

14. The system of claim 1, wherein the pet metadata comprises at least one of: breed, breed size, age, size, or weight.

15. The system of claim 14, wherein the pet metadata further comprises halitosis data and/or oral microbiota data.

16. The system of claim 13, further comprising a third user device associated with the third user, wherein the third device is configured to:
receive an electronic communication from the pet profile server, the electronic communication being generated by the pet profile server based on the contact information;
receive, from the third user via a user interface, additional pet metadata after activation of the activation code;
transmit the additional pet metadata to the pet profile server; and
receive the one or more generated reports.

17. The system of claim 16, wherein the pet metadata and the additional pet metadata comprise at least two of the following: breed, breed size, age, size, weight, body condition, shape of head, predicted size category, or predicted weight of the pet as an adult.

18. The system of claim 1, wherein the second user device is configured to:
receive, from a second user via a user interface, the activation code identified from the oral diagnostic kit, the oral diagnostic kit containing a sample collected by a first user associated with the first user device;
receive, from the second user via the user interface, the test results data obtained from a qPCR assay of the collected sample; and
transmit the activation code and the test results data to the test results server.

19. A system for providing pet health diagnostic and aftercare information, comprising:
a pet profile database storing pet metadata in association with an activation code from an oral diagnostic kit;
a quantitative polymerase chain reaction (qPCR) results database storing qPCR results in association with the activation code; and
an analytics component executed by one or more processors and configured to:
retrieve the pet metadata associated with the activation code from the pet profile database;
retrieve the qPCR results associated with the activation code from the qPCR results database;
upon determining a correlation between the pet metadata and the qPCR results based on the activation code, process the pet metadata with the qPCR results;
generate at least one diagnosis and/or at least one health recommendation based on the processed data; and
generate one or more reports based on the at least one diagnosis and/or the at least one health recommendation.

20. A computer-implemented method of providing pet health diagnostic and aftercare information, comprising:
receiving, from a pet profile server comprising a pet profile storage, pet metadata associated with an activation code, wherein the pet metadata and the activation code have been input by a first user using a first user device, the activation code identified by the first user from an oral diagnostic kit;
receiving, from a quantitative polymerase chain reaction (qPCR) results server comprising a qPCR results storage, qPCR results associated with the activation code, wherein the qPCR results are obtained from a qPCR assay of an oral sample collected from a pet;
upon determining a correlation between the pet metadata and the qPCR results based on the activation code, processing the pet metadata with the qPCR results;
generating, based on the processed data, one or more reports containing at least one diagnosis and/or at least one health recommendation; and
transmitting the one or more generated reports to at least one of the pet profile server, the first user device, or a veterinarian device.
